SEQUENCE LISTING (1 sequence)

SEQ ID NO: 1 - DNA, 601 bp, Homo sapiens
gaaaggaccagactaggtctcaccctgcagcagcctgccgccccaggactggcccagcaggctcaagagcacggtgctcattcctgggggtggggccacctctaggtggaagatggtgaaggccctacaggccccagtggggctttccaggagacctggcctttcctgtctggagcctctccagacactggggctgctcaaggtcacagcagaaggttgatggcctgcttgatgattctgcagagtcccgagtgctcgtctgttacctctcttcccctgctccagtcacgatgggcacagyctccccttgccacatccaccatctgtgcacttttacaaccccactttccatgattctcggcacagacctctggttgtgtctcatgagaatcttatctagcagtgacacagacctctctcctccatagggaacccctgggcccagctacccacctgcaggcccacccacctgccccgtgcccacaacaaccctaggcccctacactcagcctgggcgatgatagagatggcagctggccctgcctcggagccttccttggctgtgcccttggtaatcacgtcattcaggatctcccacttt

(19) United States
(12) Patent Application Publication
(10) Pub. No.: US 2011/0110930 A1
(43) Pub. Date: May 12, 2011

(54) Mitogen-Activated Protein Kinase Kinase Kinase 14 (MAP3K14) Polymorphisms As Indicators of Subject Outcome in Critically Ill Subjects

(76) Inventors: Keith R. Walley, North Vancouver (CA); James A. Russell, Vancouver (CA); Karen Patricia Mooder, Vancouver (CA); Asim Sarosh Siddiqui, Vancouver (CA)

(21) Appl. No.: 12/677,273

(22) PCT Filed: Sep. 10, 2008
(86) PCT No.: PCT/CA2008/001612

§ 371 Date: Dec. 22, 2010

Related U.S. Application Data
(60) Provisional application No. 60/935,999, filed on Sep. 10, 2007.
(60) Provisional application No. 60/960,007, filed on Sep. 11, 2007.

(51) Int. Cl. A61K 39/395; C12Q 1/68; C40B 30/04; A61K 38/02; A61K 31/56; A61K 31/573; A61P 29/00; A61P 7/02; A61P 9/10; A61P 31/00; A61P 31/18; A61P 11/00; A61P 7/06; A61P 31/10; A61P 13/12

(52) U.S. Cl. 424/133.1; 435/6; 506/9; 424/158.1; 424/142.1; 424/145.1; 424/178.1; 514/1.1; 514/169; 514/179

(57) ABSTRACT

The present application provides methods, uses, commercial packages, and kits for obtaining a prognosis for a subject having or at risk of developing an inflammatory condition and for identifying subjects having a greater benefit from treatment with an anti-inflammatory agent or an anti-coagulant agent. The method generally includes determining the genotype at position rs7222094 or a polymorphic site in linkage disequilibrium thereto for a subject for a polymorphisms in the these genes. The application also provides for methods of identifying potential subjects having an inflammatory condition who are more likely to benefit from treatment with an anti-inflammatory agent or anti-coagulant agent and to recover from the inflammatory condition. The invention also provides for methods of treating such subjects with an anti-inflammatory agent or anti-coagulant agent based on the subject's genotype.

### FIELD OF THE INVENTION

The field of the invention relates to the assessment and/or treatment of subjects with an inflammatory condition.

### BACKGROUND OF THE INVENTION

Tumour Necrosis Factor (TNF) is considered one of the most important early proinflammatory cytokines involved in host defense (Rahman M M and McFadden G Modulation of tumor necrosis factor by microbial pathogens, PLoS Pathogens, 2006 2(2):e4), (Clark I A. How TNF was recognized as a key mechanism of disease. Cytokine Growth Factor Rev. 2007 June-August; 18(3-4):335-43) while NF-kappaB (NFKB) is a fundamental transcription factor responsible for upregulating hundreds of genes involved in inflammatory processes (Ahn K S, Aggarwal B B Transcription factor NF-kappaB: a sensor for smoke and stress signals. Ann N Y Acad. Sci. 2005 (1056):218-33). A complex positive feedback loop exists between the TNF and NFKB pathways which involve the participation of a number of mediators which function to tightly regulate the expression of NFKB. Among these regulators are a series of protein kinases known as MAP kinases whose role is to activate downstream targets through the process of phosphorylation.

One of these MAP kinases is mitogen-activated protein kinase kinase kinase 14 (MAP3K14) also known as HS; NIK; HSNIK; FTDCR1B, which is an integral mediator of NFKB trafficking to the nucleus (Karin M, Ben-Neriah Y Phosphorylation meets ubiquitination: the control of NF-kappa B activity. Annu Rev Immunol. 2000; 18:621-63). In the context of infection, MAP3K14 activation of NFKB is initially induced by agents such as endotoxin that activates Toll-Like Receptors (Sabroe I, Dower S K, Whyte M K. The role of Toll-like receptors in the regulation of neutrophil migration, activation, and apoptosis. Clin Infect Dis. 2005 Nov. 15:41 Suppl 7:S421-6.). NFKB translocation to the nucleus results in the transcriptional activation of TNF which then can induce the MAP3K14 pathway again to reactivate NFKB. Thus, MAP3K14 plays a central role in the inflammatory response produced from the TNF-NFKB autoregulatory loop.

A representative human MAP3K14 mRNA sequence is listed in GenBank under accession number Y10256.

Genotype has been shown to play a role in the prediction of subject outcome in inflammatory and infectious diseases (MCGUIRE W. et al. Nature (1994) 371:508-10; NADEL S. et al. Journal of Infectious Diseases (1996) 174:878-80; MIRA J P. et al. JAMA (1999) 282:561-8; MAJETSCHAK M. et al. Ann Surg (1999) 230:207-14; STUBER F. et al. Crit. Care Med (1996) 24:381-4; STUBER F. et al. Journal of Inflammation (1996) 46:42-50; and WEITKAMP J H. et al. Infection (2000) 28:92-6). Furthermore, genotype can alter response to therapeutic interventions. Genentech's HERCEPTIN® was not effective in its overall Phase III trial but was shown to be effective in a genetic subset of subjects with human epidermal growth factor receptor 2 (HER2)-positive metastatic breast cancer. Similarly, Novartis' GLEEVEC® is only indicated for the subset of chronic myeloid leukemia subjects who carry a reciprocal translocation between chromosomes 9 and 22.

The septic inflammatory response involves counter-regulation between pro- and anti-inflammatory cytokines, pro-coagulant and fibrinolytic factors, pro-apoptotic and anti-apoptotic activity, and further counter-regulatory activity in related pathways. Altered balance of these counter-regulatory pathways leads to altered clinical outcome in subjects having an inflammatory condition, for example severe sepsis. Genetic variation between individuals is one factor that can alter the balance of these pathways and may lead to altered clinical outcome.

New therapies for severe sepsis often aim to beneficially alter this counter-regulatory balance using strategies targeting one or more of these specific pathways. A number of potential anti-inflammatory therapies focused on the TNF pathway have been investigated for the treatment of sepsis with varying degrees of success. Steroids such as hydrocortisone, cortisone, methylprednisolone and fludrocortisone are frequently used in the treatment of severe sepsis and septic shock.

### SUMMARY OF THE INVENTION

This invention is based in part on the surprising discovery that a SNP from the MAP3K14 gene is predictive or indicative of subject outcome, wherein subject outcome is the ability of the subject to recover from an inflammatory condition based on having a particular MAP3K14 genotype as compared to a subject not having that genotype.

This invention is also based in part on the surprising discovery of a MAP3K14 SNP having an association with improved prognosis or subject outcome, in subjects with an inflammatory condition. Furthermore, a SNP from MAP3K14 is provided which is useful for subject screening, as an indication of subject outcome, or for prognosis for recovery from an inflammatory condition.

This invention is also based in part on the identification of a particular nucleotide (allele) or genotype at the site of a given SNP may be associated with a decreased likelihood of recovery from an inflammatory condition (‘risk genotype’) or an increased likelihood of recovery from an inflammatory condition (‘decreased risk genotype’). Furthermore, this invention is in part based on the discovery that the genotype or allele may be predictive of increased responsiveness to the treatment of the inflammatory condition with anti-inflammatory agents or anti-coagulant agents (i.e. “adverse response genotype” (ARG) or “improved response genotype” (IRG)).

This invention is also based in part on the surprising discovery that the rs7222094 SNP is useful in predicting the response a subject with an inflammatory condition will have to treatment with anti-inflammatory agent or anti-coagulant agent. Whereby the subjects having an improved response genotype (rs7222094TT) are more likely to benefit from and have an improved response to treatment with anti-inflammatory agent or anti-coagulant agent and subjects having a adverse response genotype (rs7222094CC or rs7222094CT) are less likely to benefit from the same treatment. Furthermore, SNPs in linkage disequilibrium (LD) to rs7222094 may also be useful in predicting the response a subject with an inflammatory condition will have to treatment with anti-inflammatory agent or anti-coagulant agent.

In accordance with one aspect of the invention, methods are provided for obtaining a prognosis for a subject having, or at risk of developing, an inflammatory condition, the method including determining a genotype for the subject which includes one or more polymorphic sites in the subject's MAP3K14 gene sequence, wherein said genotype is indicative of an ability of the subject to recover from the inflammatory condition and wherein the polymorphic site is at rs7222094 or a polymorphic site in linkage disequilibrium thereto.

In accordance with a further aspect of the invention, methods are provided for selecting a group of subjects for determining the efficacy of a candidate drug known or suspected of being useful for the treatment of an inflammatory condition, the method including determining a genotype at one or more polymorphic sites in the subject's MAP3K14 gene sequence for each subject, wherein the genotype is indicative of the subject's ability to recover from the inflammatory condition and sorting subjects based on their genotype, wherein the polymorphic site rs7222094 or a polymorphic site in linkage disequilibrium thereto. The method may further include administering the candidate drug to the subjects or a subset of subjects and determining each subject's ability to recover from the inflammatory condition. The method may further include comparing subject response to the candidate drug based on genotype of the subject.

In accordance with a further aspect of the invention, methods are provided for treating an inflammatory condition in a subject in need thereof, the method including administering to the subject an anti-inflammatory agent or an anti-coagulant agent, wherein the subject has an improved response genotype in their MAP3K14 gene sequence, wherein the improved response genotype is selected from rs7222094TT or a polymorphic site in linkage disequilibrium thereto.

In accordance with a further aspect of the invention, methods are provided for treating an inflammatory condition in a subject in need thereof, the method including selectively not administering an anti-inflammatory agent or an anti-coagulant agent to the subject, wherein the subject has an adverse response genotype in their MAP3K14 gene sequence, wherein the adverse response genotype is selected from rs7222094CC or rs7222094CT or a polymorphic site in linkage disequilibrium thereto.

In accordance with a further aspect of the invention, methods are provided for selecting a subject for the treatment of an inflammatory condition with an anti-inflammatory agent or an anti-coagulant agent, including identifying a subject having an improved response genotype in their MAP3K14 gene sequence selected from rs7222094TT or a polymorphic site in linkage disequilibrium thereto, wherein the identification of a subject with the improved response genotype is predictive of increased responsiveness to the treatment of the inflammatory condition with the anti-inflammatory agent or the anti-coagulant agent.

In accordance with a further aspect of the invention, methods are provided for selecting a subject for the treatment of an inflammatory condition, including the step of identifying a subject having an adverse response genotype in their MAP3K14 gene sequence, selected from rs7222094CC or rs7222094CT or a polymorphic site in linkage disequilibrium thereto, wherein the subject is selectively not treated with an anti-inflammatory agent or an anti-coagulant agent, wherein the identification of a subject with the adverse response genotype is predictive of decreased responsiveness to the treatment of the inflammatory condition with the anti-inflammatory agent or the anti-coagulant agent.

In accordance with a further aspect of the invention, anti-inflammatory agents or an anti-coagulant agents are provided for use in the manufacture of a medicament for the treatment of an inflammatory condition, wherein the subjects treated have an improved response genotype in their MAP3K14 gene sequence, selected from rs7222094TT or a polymorphic site in linkage disequilibrium thereto.

In accordance with a further aspect of the invention, anti-inflammatory agents or an anti-coagulant agents are provided for use in the manufacture of a medicament for the treatment of an inflammatory condition, wherein the subjects treated do not have an adverse response genotype in their MAP3K14 gene sequence, selected from rs7222094CC or rs7222094CT or a polymorphic site in linkage disequilibrium thereto.

In accordance with another aspect of the invention, methods are provided for predicting whether a subject having, or at risk of developing, an inflammatory condition will have relatively greater or fewer days alive, and days alive and free from organ dysfunction, including one or more conditions selected from the following: cardiovascular dysfunction, respiratory dysfunction, coagulation dysfunction, liver failure, and the use of vital life support including use of vasopressors, inotropes, ventilation and renal support.

In accordance with a further aspect of the invention, methods are provided for identifying a polymorphism in a MAP3K14 gene sequence that correlates with prognosis of recovery from an inflammatory condition, the method including: (a) obtaining MAP3K14 gene sequence infoiniation from a group of subjects having an inflammatory condition; (b) identifying at least one polymorphic nucleotide position in the MAP3K14 gene sequence in the subjects; (c) determining a genotypes at the polymorphic site for individual subjects in the group; (d) determining recovery capabilities of individual subjects in the group from the inflammatory condition; and (e) correlating the genotypes determined in step (c) with the recovery capabilities determined in step (d) thereby identifying said MAP3K14 gene sequence polymorphisms that correlate with recovery.

In accordance with a further aspect of the invention, a kit is provided for deteimining a genotype at a defined nucleotide position within a polymorphic site in MAP3K14 gene sequence in a subject to provide a prognosis of the subject's ability to recover from an inflammatory condition, the kit including: a restriction enzyme capable of distinguishing alternate nucleotides at the polymorphic site; or a labeled oligonucleotide having sufficient complementary to the polymorphic site so as to be capable of hybridizing distinctively to said alternate. The kit may further include an oligonucleotide or a set of oligonucleotides operable to amplify a region including the polymorphic site. The kit may further include a polymerization agent. The kit may further include instructions for using the kit to determine genotype.

In accordance with another aspect of the invention, there is provided a use of one or more anti-inflammatory agents or anti-coagulant agents in the manufacture of a medicament for the treatment of an inflammatory condition, wherein the subjects treated do not have an adverse response polymorphism in their rs7222094 sequence or a polymorphic site in linkage disequilibrium thereto.

In accordance with another aspect of the invention, there is provided a commercial package containing, as active pharmaceutical ingredient, one or more anti-inflammatory agents or anti-coagulant agents, or a pharmaceutically acceptable salt thereof, together with instructions for its use for the curative or prophylactic treatment of an inflammatory condition in a subject, wherein the subject treated has an improved response polymorphism in their MAP3K14 gene sequence.

In accordance with another aspect of the invention, there is provided a commercial package containing, as active pharmaceutical ingredient, use of one or more anti-inflammatory agents or anti-coagulant agents, or a pharmaceutically acceptable salt thereof, together with instructions for its use for the curative or prophylactic treatment of an inflammatory condition in a subject, wherein the subject treated does not have an adverse response polymorphism in their MAP3K14 gene sequence.

A subject having an rs7222094CC or rs7222094CT genotype or a polymorphic site in linkage disequilibrium thereto may be at risk of having a poor outcome from the inflammatory condition. A subject having an rs7222094TTgenotype or a polymorphic site in linkage disequilibrium thereto may be at reduced risk of having a poor outcome from the inflammatory condition.

Genotype may be determined using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data.

The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis. The inflammatory condition may also be selected from one or more of: SIRS, sepsis, septic shock and cardiogenic shock.

The anti-inflammatory agent or the anti-coagulant agent may be a TNF inhibitor. The TNF inhibitor may be selected from one or more of an antagonist of TNF, an aptamer that binds to TNF, an aptamer that binds to a TNF receptor, a soluble TNF receptor, an anti-TNF antibody, and an anti-TNF antibody fragment. The anti-TNF antibody, and the anti-TNF antibody fragment may be selected from one or more of the following: infliximab; adalumimab; ovine polyclonal anti-TNF Fab fragment; certolizumab; and afelimomab. The soluble TNF receptor may be selected from one or more of: etanercept; lenerecept; and PEG-p55sTNF-receptor I monomer.

The anti-inflammatory agent or the anti-coagulant agent may be a steroid. The steroid may be selected from one or more of corticosteroids, cortisone, hydrocortisone, dexamethasone, fludrocortisone, triamcinolone, prednisone, methylprednisolone and prednisolone.

Optionally, the method or use may further include determining the subject's APACHE II score as an assessment of subject risk. The method or use may further include determining the number of organ system failures for the subject as an assessment of subject risk. The subject's APACHE II score may be indicative of an increased risk when ≧25.2 or more organ system failures may be indicative of increased subject risk.

The improved response genotype may be found at one or more of the following polymorphic sites: rs7222094; or a polymorphic site in linkage disequilibrium thereto.

The improved response genotype may be selected from rs7222094TT or a polymorphic site in linkage disequilibrium thereto. The adverse response genotype may be selected from one or more of the following: rs7222094CC or rs7222094CT; or a polymorphic site in linkage disequilibrium thereto.

The subject having one or more improved response genotypes may be selectively administered the anti-inflammatory agent or anti-coagulant agent. The subject having one or more adverse response genotypes may be selectively not administered the anti-inflammatory agent or anti-coagulant agent. The genotype may be determined using a nucleic acid sample from the subject. The method or use may further include obtaining the nucleic acid sample from the subject.

In accordance with another aspect of the invention, there are provided two or more oligonucleotides or peptide nucleic acids of about 10 to about 400 nucleotides that hybridize specifically to a sequence contained in a human target sequence consisting of a subject's MAP3K14 gene sequence, a complementary sequence of the target sequence or RNA equivalent of the target sequence and wherein the oligonucleotides or peptide nucleic acids are operable in determining the presence or absence of two or more polymorphism(s) or in their MAP3K14 gene sequence selected from of the following polymorphic site: rs7222094; or one or more polymorphic sites in linkage disequilibrium thereto.

In accordance with another aspect of the invention, there are provided two or more oligonucleotides or peptide nucleic acids selected from the group including of: (a) an oligonucleotide or peptide nucleic acid that hybridizes under high stringency conditions to a nucleic acid molecule including SEQ ID NO:1 having a T at position 301 but not to a nucleic acid molecule including SEQ ID NO:1 having a C at position 301; or peptide nucleic acid capable of hybridizing under high stringency conditions to a nucleic acid molecule including a first allele for a given polymorphism selected from a polymorphism in linkage disequilibrium thereto; and (v) an oligonucleotide or peptide nucleic acid capable of hybridizing under high stringency conditions to a nucleic acid molecule including the second allele for a given polymorphism selected from a polymorphism in linkage disequilibrium thereto but not capable of hybridizing under high stringency conditions to a nucleic acid molecule including the first allele for the given polymorphism in linkage disequilibrium thereto.

In accordance with another aspect of the invention, there is provided an array of oligonucleotides or peptide nucleic acids attached to a solid support, the array including two or more of the oligonucleotides or peptide nucleic acids, provided that one is rs7222094 or a polymorphic sites in linkage disequilibrium thereto.

In accordance with another aspect of the invention, there is provided a composition including an addressable collection of two or more oligonucleotides or peptide nucleic acids, the two or more oligonucleotides or peptide nucleic acids selected from the oligonucleotides or peptide nucleic acids, provided that one is rs7222094 or a polymorphic sites in linkage disequilibrium thereto.

In accordance with another aspect of the invention, there is provided a composition including an addressable collection of two or more oligonucleotides or peptide nucleic acids, the two or more oligonucleotides or peptide nucleic acids including two or more nucleic acid molecules set out in SEQ ID NO:1 or compliments, fragments, variants, or analogs thereof.

In accordance with another aspect of the invention, there is provided a composition including an addressable collection of two or more oligonucleotides or peptide nucleic acids, the two or more oligonucleotides or peptide nucleic acids including two or more nucleic acid molecules set out herein or compliments, fragments, variants, or analogs thereof, provided that one is rs7222094 or a polymorphic sites in linkage disequilibrium thereto.

The oligonucleotides or peptide nucleic acids described herein may further include one or more of the following: a detectable label; a quencher; a mobility modifier; a contiguous non-target sequence situated 5′ or 3′ to the target sequence or 5′ and 3′ to the target sequence.

The oligonucleotides or peptide nucleic acids may further include one or more of the following: a detectable label; a quencher; a mobility modifier; a contiguous non-target sequence situated 5′ or 3′ to the target sequence or 5′ and 3′ to the target sequence. The oligonucleotides or peptide nucleic acids may alternatively be of about 10 to about 400 nucleotides, about 15 to about 300 nucleotides. The oligonucleotides or peptide nucleic acids may alternatively be of about 20 to about 200 nucleotides, about 25 to about 100 nucleotides. The oligonucleotides or peptide nucleic acids may alternatively be of about 20 to about 80 nucleotides, about 25 to about 50 nucleotides. Two or more oligonucleotides or peptide nucleic acids may include 3 or more; 4 or more; 5 or more; 6 or more; 7 or more; 8 or more; 9 or more; 10 or more; 11 or more; 12 or more; 13 or more; 14 or more; 15 or more; 16 or more; 17 or more; 18 or more; 19 or more; or 20 or more. A determination of whether a site is in linkage disequilibrium (LD) with another site may be determined based on an absolute r² value or D′ value. When evaluating loci for LD those sites within a given population having a high degree of linkage disequilibrium (for example an absolute value for D′ of ≧0.5 or r²≧0.5) are potentially useful in predicting the identity of an allele of interest (for example associated with the condition of interest). A high degree of linkage disequilibrium may be represented by an absolute value for D′ of ≧0.6 or r²≧0.6. Alternatively, a higher degree of linkage disequilibrium may be represented by an absolute value for D′ of ≧0.7 or r²≧0.7 or by an absolute value for D′ of ≧0.8 or r²≧0.8. Additionally, a high degree of linkage disequilibrium may be represented by an absolute value for D′ of ≧0.85 or r²≧0.85 or by an absolute value for D′ of ≧0.9 or r²≧0.9.

Sequence variations may be assigned to a gene if mapped within 2 kb or more of an mRNA sequence feature. In particular, such a sequence may extend many kilobases (kb) from the MAP3K14 gene sequence and into neighbouring genes, where the LD within a region is strong.

### BRIEF DESCRIPTION OF THE DRAWINGS

FIG. 1 shows a Kaplan-Meier curve for a cohort of Caucasian Subjects with severe sepsis by genotype of mitogen-activated protein kinase kinase kinase 14 (MAP3K14) rs7222094 (CC=solid, CT=dashed, TT=dot).

FIG. 2 shows a Kaplan-Meier curve for a cohort of Subjects of all ethnicities with severe sepsis by genotype of mitogen-activated protein kinase kinase kinase 14 (MAP3K14) rs7222094 (CC=solid, CT=dashed, TT=dot).

FIG. 3 shows a histogram of 28-day survival for a cohort of subjects of all ethnicities with severe sepsis by genotype of mitogen-activated protein kinase kinase kinase 14 (MAP3K14) rs7222094 in steroid treated individuals and controls.

### DETAILED DESCRIPTION OF THE INVENTION

### 1. Definitions

In the description that follows, a number of terms are used extensively, the following definitions are provided to facilitate understanding of the invention.

“Steroids” or “steroid” as used herein include, but are not limited to, corticosteroids, cortisone, hydrocortisone, dexamethasone, fludrocortisone, triamcinolone, prednisone, methylprednisolone and prednisolone.

“TNF Inhibitor” or “TNF Inhibitors” as used herein includes any molecule that interferes with or blocks the synthesis, production, processing, transcription or the biological activity or biological effect of TNF, including derivatives, variants, analogues, non-peptidyl analogues and prodrugs thereof, metabolites thereof, isomers thereof, combination of isomers thereof, conjugates thereof, salts thereof, or pharmaceutical composition of any of the preceding. Such inhibitors may be capable of binding to or interacting with TNF or a TNF receptor or to another protein or gene in the TNF pathway that indirectly exerts an effect on TNF. TNF inhibitors may be used alone or in combination with other TNF inhibitors or other medications. TNF inhibitors may be synthesized, recombinant or purified. TNF inhibitors may be small molecules, peptides, proteins, or nucleic acids. Examples of TNF inhibitors may include, but are not limited to antagonists of TNF, antibodies or other molecules such as aptamers that bind to TNF or TNF receptors, and soluble TNF receptors, Anti-TNF antibodies, include, but are not limited to, infliximab (Remicade®; mouse-human chimeric anti-humanTNF monoclonal antibody), adalumimab (Humira®; fully human anti-human TNF monoclonal antibody), ovine polyclonal anti-TNF Fab fragment (CytoFab®), certolizumab (CDP870; polyethylene glycol (PEG)ylated anti-TNF antibody fragment), and afelimomab (Segard®; anti-TNF monoclonal antibody fragment). Soluble TNF receptors, include, but are not limited to, etanercept (Enbrel®; p75sTNF-receptor II dimer), lenerecept (p55sTNF receptor I fused to IgG1 heavy chain fragment), and PEG-p55sTNF-receptor I monomer.

“Antibodies” or “Antibody” as used herein include without limitation recombinant, monoclonal and polyclonal antibodies, antibody fragments, such as Fab and Fab′ Fab′2 and Fv fragments, single chain antibodies, chimeric bispecific and heteroantibodies, as well as antibodies that are conjugated to other molecules such as polyethylene glycol (PEG),

“Anti-inflammatory agent(s)” or “anti-coagulant agent(s)” as used herein means any agent that has the ability to interfere with or block inflammatory or coagulant processes when administered to a subject. Anti-inflammatory agents include, but are not limited to: steroids, TNF inhibitors, activated protein C (or protein C like compounds; protein S or a protein S like drugs; a factor Xa inhibitors such as tissue factor pathway inhibitor (TFPI) (for example TIFACOGIN™-alpha [Chiron] and the like) or monoclonal antibodies against tissue factor (TF); serine protease inhibitors (for example antithrombin III); platelet activating factor hydrolase; PAF-AH enzyme analogues; tissue plasminogen activating factor (tPA); thrombomodulin (including analogs and variants thereof); and heparin.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). A “Nucleotide” is generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, while a nucleotide represented by the symbol D may be either A or G or T, while a nucleotide represented by the symbol B may be either G or C or T, and a nucleotide represented by the symbol H may be either A or C or T.

A “polymorphic site” or “polymorphism site” or “polymorphism” or “single nucleotide polymorphism site” (SNP site) or single nucleotide polymorphism” (SNP) as used herein is the locus or position with in a given sequence at which divergence occurs. A “polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, introns or untranslated regions) of genes. Polymorphisms may occur at a single nucleotide site (SNPs) or may involve an insertion or deletion as described herein.

A “risk genotype” as used herein refers to an allelic variant (genotype) at one or more polymorphic sites within the MAP3K14 gene sequences described herein as being indicative of a decreased likelihood of recovery from an inflammatory condition or an increased risk of having a poor outcome. The risk genotype may be determined for either the haploid genotype or diploid genotype, provided that at least one copy of a risk allele is present. Risk genotype may be an indication of an increased risk of not recovering from an inflammatory condition. Subjects having one copy (heterozygotes) or two copies (homozygotes) of the risk allele (for example rs7222094 CT, rs7222094 CC respectively) are considered to have the “risk genotype” even though the degree to which the subjects risk of not recovering from an inflammatory condition may increase, depending on whether the subject is a homozygote rather than a heterozygote. Such “risk alleles” or “risk genotypes” may be selected from the following: rs7222094 CT; rs7222094 CC; rs7222094 C or a polymorphic site in linkage disequilibrium thereto.

A “decreased risk genotype” or “reduced risk genotype” as used herein refers to an allelic variant (genotype) at one or more polymorphic sites within the MAP3K14 gene sequences described herein as being indicative of an increased likelihood of recovery from an inflammatory condition or a decreased risk of having a poor outcome. The decreased risk genotype may be determined for either the haploid genotype or diploid genotype, provided that at least one copy of a risk allele is present. Decreased risk genotype may be an indication of an increased likelihood of recovering from an inflammatory condition. Subjects having one copy (heterozygotes) or two copies (homozygotes) of the decreased risk allele (for example rs7222094 CT; rs7222094 TT) are considered to have the “decreased risk genotype” even though the degree to which the subjects risk of not recovering from an inflammatory condition may increase, depending on whether the subject is a homozygote rather than a heterozygote. Such “decreased risk alleles” or “decreased risk genotypes” or “reduced risk genotypes” may be selected from the following: rs7222094 TT; or a polymorphic site in linkage disequilibrium thereto.

An “improved response genotype” (IRG) or improved response polymorphic variant (IRP) as used herein refers to an allelic variant or genotype at one or more polymorphic sites within the MAP3K14 polymorphisms as described herein as being predictive of a subject's improved survival in response to treatment with steroids or other anti-inflammatory agents or anti-coagulant agents (for example rs7222094 TT), or a polymorphic site in linkage disequilibrium thereto.

An “adverse response genotype” (ARG) or adverse response polymorphic variant as used herein refers to an allelic variant or genotype at one or more polymorphic sites within the MAP3K14 polymorphisms as described herein as being predictive of a subject's decreased survival in response to treatment with steroids or other anti-inflammatory agents or anti-coagulant agents (for example rs7222094 CC or rs7222094 CT), or a polymorphic site in linkage disequilibrium thereto. Subjects having a ARG are preferably selected for treatments not involving steroid administration.

A “clade” is a group of haplotypes that are closely related phylogenetically. For example, if haplotypes are displayed on a phylogenetic (evolutionary) tree a clade includes all haplotypes contained within the same branch.

The pattern of a set of markers along a chromosome is referred to as a “Haplotype”. Accordingly, groups of alleles on the same small chromosomal segment tend to be transmitted together. Haplotypes along a given segment of a chromosome are generally transmitted to progeny together unless there has been a recombination event. Absence of a recombination event, haplotypes can be treated as alleles at a single highly polymorphic locus for mapping.

As used herein “haplotype” is a set of alleles of closely linked loci on a chromosome that tend to be inherited together. Such allele sets occur in patterns, which are called haplotypes. Accordingly, a specific SNP or other polymorphism allele at one SNP site is often associated with a specific SNP or other polymorphism allele at a nearby second SNP site or other polymorphism site. When this occurs, the two SNPs or other polymorphisms are said to be in LD because the two SNPs or other polymorphisms are not just randomly associated (i.e. in linkage equilibrium).

In general, the detection of nucleic acids in a sample depends on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High-stringency hybridization is relied upon for the success of numerous techniques routinely performed by molecular biologists, such as high-stringency PCR, DNA sequencing, single strand conformational polymorphism analysis, and in situ hybridization. In contrast to Northern and Southern hybridizations, these aforementioned techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

“Oligonucleotides” as used herein are variable length nucleic acids, which may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI D A. et al. Proc Nat Acad Sci (1995) 92(17):7912-5; MCGALL G. et al. PNAS (1996) 93(24):13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

An “addressable collection” as used herein is a combination of nucleic acid molecules or peptide nucleic acids capable of being detected by, for example, the use of hybridization techniques or by any other means of detection known to those of ordinary skill in the art. A DNA microarray would be considered an example of an “addressable collection”.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like SNPs, are often useful in tracking meiotic recombination events as positional markers on chromosomes.

Furthermore, the preferential occurrence of a disease gene in association with specific alleles of linked markers, such as SNPs or other polymorphisms, is called “Linkage Disequilibrium” (LD). This sort of disequilibrium generally implies that most of the disease chromosomes carry the same mutation and the markers being tested are relatively close to the disease gene(s).

For example, in SNP-based association analysis and LD mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs may be tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For an LD based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs) and short tandem repeats (STRs).

In population genetics linkage disequilibrium refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as position 201 of SEQ ID NO:1 (see Akey, J. et al. Eur J Hum Genet (2001) 9:291-300; and Zhang, K. et al. (2002). Am J Hum Genet. 71:1386-1394). This viewpoint is further substantiated by Khoury et al. ((1993). Fundamentals of Genetic Epidemiology. New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for r²≧0.5) are potentially useful in predicting the identity of an allele of interest (i.e. associated with the condition of interest). A high degree of linkage disequilibrium may be represented by an absolute value for r²≧0.6. Alternatively, a high degree of linkage disequilibrium may be represented by an absolute value for r²≧0.7 or by an absolute value for r²≧0.8. Additionally, a high degree of linkage disequilibrium may be represented by an absolute value for r²≧0.85 or by an absolute value for r²≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably.

LD is useful for genotype-phenotype association studies. For example, if a specific allele at one SNP site (e.g. “A”) is the cause of a specific clinical outcome (e.g. call this clinical outcome “B”) in a genetic association study then, by mathematical inference, any SNP (e.g. “C”) which is in significant LD with the first SNP, will show some degree of association with the clinical outcome. That is, if A is associated (˜) with B, i.e. A˜B and C˜A then it follows that C˜B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on LD between A and C.

Until the mechanism underlying the genetic contribution to a specific clinical outcome is fully understood, LD helps identify potential candidate causal SNPs and also helps identify a range of SNPs that may be clinically useful for prognosis of clinical outcome or of treatment effect. If one SNP within a gene is found to be associated with a specific clinical outcome, then other SNPs in LD will also have some degree of association and therefore some degree of prognostic usefulness.

By way of prophetic example, if multiple polymorphisms were tested for individual association with an improved response to steroid administration in our SIRS/sepsis/septic shock cohort of ICU subjects, wherein the multiple polymorphisms had a range of LD with MAP3K14 rs7222094 and it was assumed that rs7222094 was the causal polymorphism, and we were to order the polymorphisms by the degree of LD with rs7222094, we would expect to find that polymorphisms with high degrees of LD with rs7222094 would also have a high degree of association with this specific clinical outcome. As LD decreased, we would expect the degree of association of the polymorphism with an improved response steroid administration to also decrease. It follows that any polymorphism, whether already discovered or as yet undiscovered, that is in LD with one of the improved response genotypes described herein will likely be a predictor of the same clinical outcomes that rs7222094 is a predictor of. The similarity in prediction between this known or unknown polymorphism and rs7222094 would depend on the degree of LD between such a polymorphism and rs7222094.

TABLE 1A
Polymorphisms in the MAP3K14 gene genotyped in a cohort
of critically ill Subjects with severe sepsis.

| Polymor- | | | March 2006 | | Minor |
|---|---|---|---|---|---|
| phism | Official | | Chromosomal | | Allele |
| Name | Gene | | position | Minor | Fre- |
| (Alleles) | Name | rs# | (Build 36) | allele | quency |
| MAP3K14 | mitogen- | rs7222094 | 40723436 | C | 0.475 |
| rs7222094 | activated | | | | |
| (C/T) | protein | | | | |
| | kinase kinase | | | | |
| | kinase 14 | | | | |

Minor Allele Frequencies (MAFs) for Caucasians were taken from NCBI (http://www.ncbi.nlm.nih.gov/).

It will be appreciated by a person of skill in the art that linked polymorphic sites and combined polymorphic sites may be determined. A haplotype of MAP3K14 gene can be created by assessing polymorphisms in MAP3K14 gene in normal subjects using a program that has an expectation maximization algorithm (i.e. PHASE). A constructed haplotype of the MAP3K14 gene may be used to find combinations of SNPs that are in LD with the SNP identified herein. Accordingly, the haplotype of an individual could be determined by genotyping other SNPs or other polymorphisms that are in LD with the SNP identified herein. Single polymorphic sites or combined polymorphic sites in LD may also be genotyped for assessing subject response to steroid treatment.

It will be appreciated by a person of skill in the art that the numerical designations of the positions of polymorphisms within a sequence are relative to the specific sequence. Also the same positions may be assigned different numerical designations depending on the way in which the sequence is numbered and the sequence chosen, as illustrated by the alternative numbering of the equivalent polymorphism (rs7222094), whereby the same polymorphism identified C/T at chromosomal position 40723436 (Build 36) corresponds to position 301 of SEQ ID NO:1. Furthermore, sequence variations within the population, such as insertions or deletions, may change the relative position and subsequently the numerical designations of particular nucleotides at and around a polymorphic site.

Polymorphic sites in SEQ ID NO:1 are identified by their variant designation (i.e. M, W, Y, S, R, K, V, B, D, H or by “−” for a deletion, a “+” or for example “G” etc. for an insertion).

An “rs” prefix designates a SNP in the database is found at the NCBI SNP database (http://www.ncbi.nlm.nih.govientrez/query.fcgi?db=Snp). The “rs” numbers are the NCBI|rsSNP ID form.

TABLE 1B below shows the flanking sequences
for a MAP3K14 gene SNP and providing the rs
designation and corresponding SEQ ID NO
designation. The polymorphism is at
position 301 within the flanking sequence,
and identified in bold and underlined.

| | | SEQ | |
|---|---|---|---|
| | | ID | |
| GENE | SNP | NO: | FLANKING SEQUENCE |
| MAP3K14 | rs7222094 | 1 | GAAAGGACCAGACTAGGTCTCACCCT |
| | | | GCAGCAGCCTGCCGCCCCAGGACTGG |
| | | | CCCAGCAGGCTCAAGAGCACGGTGCT |
| | | | CATTCCTGGGGGTGGGGCCACCTCTA |
| | | | GGTGGAAGATGGTGAAGGCCCTACAG |
| | | | GCCCCAGTGGGGCTTTCCAGGAGACC |
| | | | TGGCCTTTCCTGTCTGGAGCCTCTCC |
| | | | AGACACTGGGGCTGCTCAAGGTCACA |
| | | | GCAGAAGGTTGATGGCCTGCTTGATG |
| | | | ATTCTGCAGAGTCCCGAGTGCTCGTC |
| | | | TGTTACCTCTCTTCCCCTGCTCCAGT |
| | | | CACGATGGGCACAGYCTCCCCTTGCC |
| | | | ACATCCACCATCTGTGCACTTTTACA |
| | | | ACCCCACTTTCCATGATTCTCGGCAC |
| | | | AGACCTCTGGTTGTGTCTCATGAGAA |
| | | | TCTTATCTAGCAGTGACACAGACCTC |
| | | | TCTCCTCCATAGGGAACCCCTGGGCC |
| | | | CAGCTACCCACCTGCAGGCCCACCCA |
| | | | CCTGCCCCGTGCCCACAACAACCCTA |
| | | | GGCCCCTACACTCAGCCTGGGCGATG |
| | | | ATAGAGATGGCAGCTGGCCCTGCCTC |
| | | | GGAGCCTTCCTTGGCTGTGCCCTTGG |
| | | | TAATCACGTCATTCAGGATCTCCCAC |
| | | | TTT |

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the SNP of interest.

A “genotype” is defined as the genetic constitution of an organism, usually in respect to one gene or a few genes or a region of a gene relevant to a particular context (i.e. the genetic loci responsible for a particular phenotype).

A “phenotype” is defined as the observable characters of an organism. In gene association studies, the genetic model at a given locus can change depending on the selection pressures (i.e., the environment), the population studied, or the outcome variable (i.e., the phenotype).

One observation would be seen in a gene association study with the hemoblobin, beta gene (HBB) with mortality as the primary outcome variable. A mutation in the HBB gene, which normally produces the beta chain subunit of hemoglobin (B allele), results in an abnormal beta chain called hemoglobin S(S allele; Allison A (1955) Cold Spring Harbor Symp. Quant. Biol. 20:239-255). Hemoglobin S results in abnormal sickle-shaped red blood cells which lead to anemia and other serious complications including death. In the absence of malaria, a gene association study with the HBB gene would suggest a codominant model (survival(BB)>survival (BS)>survival (SS)). However, in the presence of marlaria, a gene association study with the HBB gene would suggest a heterozygote advantage model (survival(BB)<survival(BS)>survival(SS)).

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins” or “I”) of a nucleotide relative to a reference allele. Furthermore, a person of skill in the art would appreciate that an insertion or deletion within a given sequence could alter the relative position and therefore the position number of another polymorphism within the sequence. Furthermore, although an insertion or deletion may by some definitions not qualify as a SNP as it may involve the deletion of or insertion of more than a single nucleotide at a given position, as used herein such polymorphisms are also called SNPs as they generally result from an insertion or deletion at a single site within a given sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature>38° C. or <36° C., B) heart rate>90 beats per minute, C) respiratory rate>20 breaths per minute, or PaCO₂<32 mm Hg or the need for mechanical ventilation, and D) white blood cell count>12,000 per mm³ or <4,000 mm³. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication.

Subject outcome or prognosis as used herein refers the ability of a subject to recover from an inflammatory condition and may be used to determine the efficacy of a treatment regimen, for example the administration of a steroid. An inflammatory condition, may be selected from the group consisting of: sepsis, septicemia, pneumonia, septic shock, systemic inflammatory response syndrome (SIRS), Acute Respiratory Distress Syndrome (ARDS), cardiogenic shock, disseminated intravascular coagulation (DIC), acute kidney injury, acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome, prognosis, or response of a subject to steroid or other anti-inflammatory or anti-coagulant agent administration may be accomplished by various methods. For Example, an “APACHE II” score is defined as Acute Physiology And Chronic Health Evaluation and herein was calculated on a daily basis from raw clinical and laboratory variables. Vincent et al. (Vincent J L. Ferreira F. Moreno R. 2000 Crit. Care Clin. 16:353-366) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials”.

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO₂/FiO₂ ratio is less than 300 mmHg, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28-day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine or phenylephrine). Days alive and free of an International Normalized Ratio (INR)>1.5 are days that a person is alive and does not have an INR>1.5.

TABLE 2A
Brussels Organ Dysfunction Scoring System

| | Free of Organ Dysfunction | | Clinically Significant Organ Dysfunction | | |
|---|---|---|---|---|---|
| ORGANS | Normal | Mild | Moderate | Severe | Extreme |
| DAF ORGAN | 1 | | 0 | | |
| DYSFUNCTION | | | | | |
| SCORE | | | | | |
| Cardiovascular | >90 | ≦90 | ≦90 | ≦90 plus | ≦90 plus |
| Systolic BP | | Responsive | Unresponsive | pH ≦7.3 | pH ≦7.2 |
| (mmHg) | | to fluid | to fluid | | |
| Pulmonary | >400 | 400-301 | 300-201 | 200-101 | ≦100 |
| Pₐo₂/F_{I}o₂ (mmHg) | | | Acute lung | ARDS | Severe ARDS |
| | | | injury | | |
| Renal | <1.5 | 1.5-1.9 | 2.0-3.4 | 3.5-4.9 | ≧5.0 |
| Creatinine (mg/Dl) | | | | | |
| Hepatic | <1.2 | 1.2-1.9 | 2.0-5.9 | 6.0-11.9 | ≧12 |
| Bilirubin (mg/dL) | | | | | |
| Hematologic | >120 | 120-81 | 80-51 | 50-21 | ≦20 |
| Platelets (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, Mar. 12-14, 1994.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), cardiogenic shock, disseminated intravascular coagulation (DIG), acute kidney injury, acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulonephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition or is to be administered an anti-inflammatory agent or an anti-coagulant agent, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material, particularly, genetic material containing the sequence or sequences of interest. Many methods are known in the art for collecting biological samples and extracting genetic material from those samples. Genetic material can be extracted from blood, tissue, hair and other biological material. There are many methods known to isolate DNA and RNA from biological material. Typically, DNA may be isolated from a biological sample when first the sample is lysed and then the DNA is separated from the lysate according to any one of a variety of multi-step protocols, which can take varying lengths of time. DNA isolation methods may involve the use of phenol (Sambrook, J. et al., “Molecular Cloning”, Vola 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR), Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of a mutation in a MAP3K14 gene sequence, as described in TABLES 1A-B. The sequence of interest may also include other mutations, or may also contain some of the sequence surrounding the mutation of interest.

Detection or determination of a nucleotide identity, or the presence of one or more single nucleotide polymorphism(s) (SNP typing), may be accomplished by any one of a number methods or assays known in the art. Many DNA typing methodologies are useful for use in the detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

Alternatively, an invasive cleavage method requires an oligonucleotide called an Invader™ probe and sequence-specific probes to anneal to the target DNA with an overlap of one nucleotide. When the sequence-specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

The Illumina Golden Gate™ Assay uses a combined oligonucleotide ligation assay/allele-specific hybridization approach (SHEN R et al Mutat Res 2005573:70-82). The first series of steps involve the hybridization of three oligonucleotides to a set of specific target SNPs; two of these are fluorescently-labelled allele-specific oligonucleotides (ASOs) and the third a locus-specific oligonucleotide (LSO) binding 1-20 by downstream of the ASOs. A second series of steps involve the use of a stringent polymerase with high 3′ specificity that extends only oligonucleotides specifically matching an allele at a target SNP. The polymerase extends until it reaches the LSO. Locus-specificity is ensured by requiring the hybridization of both the ASO and LSO in order that extension can proceed. After PCR amplification with universal primers, these allele-specific oligonucleotide extension products are hybridized to an array which has multiple discretely tagged addresses (in this case 1536 addresses) which match an address embedded in each LSO. Fluorescent signals produced by each hybridization product are detected by a bead array reader from which genotypes at each SNP locus may be ascertained.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP system.

Mutation detection methods may include but are not limited to the following:

Restriction Fragment Length Polymorphism (RFLP) strategy—An RFLP gel-based analysis can be used to indicate the presence or absence of a specific mutation at polymorphic sites within a gene. Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. Finally, if both the higher molecular weight band and the two lower molecular weight bands are visible then the DNA sample contained both polymorphisms, and therefore the DNA sample, and by extension the subject providing the DNA sample, was heterozygous for this polymorphism;

For example the Maxam-Gilbert technique for sequencing (MAXAM A M. and GILBERT W. Proc. Natl. Acad. Sci. USA (1977) 74(4):560-564) involves the specific chemical cleavage of terminally labeled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et al., (Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific teiminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Qβ replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4):1760-1764). Other methods include those of Donis-Keller et al. (1977, Nucl. Acids Res. 4:2527-2538), SIMONCSITS A. et al. (Nature (1977) 269(5631):833-836), AXELROD V D. et al. (Nucl. Acids Res. (1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-terminator incorporation with fluorescent polarization-detection (TDI-FP) method is described by FREEMAN B D. et al. (J Mol Diagnostics (2002) 4(4):209-215) for large scale screening;

Oligonucleotide ligation assay (OLA) is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2):123-9; IANNONE M A. et al. Cytometry (2000) 39(2):131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8);

Polymerase proofreading methods are used to determine SNPs identities, as described in WO 0181631;

Detection of single base pair DNA mutations by enzyme-amplified electronic transduction is described in PATOLSKY F et al. Nat. Biotech. (2001) 19(3):253-257;

Gene chip technologies are also known for single nucleotide polymorphism discrimination whereby numerous polymorphisms may be tested for simultaneously on a single array (EP 1120646 and GILLES P N. et al. Nat. Biotechnology (1999) 17(4):365-70);

Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43:1151-1158; LITTLE DP. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

Genotyping at single nucleotide polymorphisms may be done using dideoxy single-base extension, in which an unlabelled complementary primer binds to a region of the gene immediately upstream of the SNP, and the primer is extended (in the presence of labeled ddNTPs and DNA polymerase) by one nucleotide at its 3′ end (for example, the Applied Biosystems SNaPshot™ kit).

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites.

Once the identity of a polymorphism(s) is determined or detected an indication may be obtained as to subject response to steroid administration based on the genotype (the nucleotide at the position) of the polymorphism of interest. In the present invention, polymorphisms in MAP3K14 gene sequence, are used to predict a subject's response to steroid treatment. Methods for predicting a subject's response to steroid treatment may be useful in making decisions regarding the administration of a steroid.

Methods of treatment of an inflammatory condition in a subject having an improved response genotype in the MAP3K14 gene are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, central nervous system, coagulation [INR>1.5], renal and/or hepatic).

As described above genetic sequence information or genotype information may be obtained from a subject wherein the sequence information contains one or more polymorphic sites in the MAP3K14 gene sequence. Also, as previously described the sequence identity of one or more polymorphisms in the MAP3K14 gene sequence of one or more subjects may then be detected or determined. Furthermore, subject response to administration of a steroid may be assessed as described above. For example, the APACHE II scoring system or the Brussels score may be used to assess a subject's response to treatment by comparing subject scores before and after treatment. Once subject response has been assessed, subject response may be correlated with the sequence identity of one or more polymorphism(s). The correlation of subject response may further include statistical analysis of subject outcome scores and polymorphism(s) for a number of subjects.

Methods of treatment of an inflammatory condition in a subject having one or more of the risk genotypes in MAP3K14 (or a SNP in linkage disequilibrium thereto) associated with improved response to a therapeutic agent are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, central nervous system, coagulation [INR>1.5], renal and/or hepatic).

As described above genetic sequence information or genotype information may be obtained from a subject wherein the sequence information contains one or more single nucleotide polymorphic sites in MAP3K14 sequences. Also, as previously described the sequence identity of one or more single nucleotide polymorphisms in the MAP3K14 sequences of one or more subjects may then be detected or determined. Furthermore, subject outcome or prognosis may be assessed as described above, for example the APACHE II scoring system or the Brussels score may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis of subject outcome scores and polymorphism(s) for a number of subjects.

### 3. Analytical Methods

### Patient Cohort Selection

a. Intensive Care Unit (ICU) Cohort Inclusion Criteria

All subjects admitted to the ICU of St. Paul's Hospital (SPH) were screened for study inclusion. SPH ICU is a mixed medical-surgical ICU in a tertiary care, university-affiliated teaching hospital. Subjects were included in the study if they met at least two out of four SIRS criteria: 1) fever (>38° C.) or hypothermia (<36° C.), 2) tachycardia (>90 beats/minute), 3) tachypnea (>20 breaths/minute), PaCO₂<32 mm Hg, or need for mechanical ventilation, and 4) leukocytosis (total leukocyte count>12,000 mm³) or leukopenia (<4,000 mm³). Subjects were included in the analysis if they met the diagnostic criteria for septic shock (sepsis and cardiovascular dysfunction (as defined by Brussels scoring system) and one other organ dysfunction) on admission to the ICU. Subjects were excluded if blood could not be obtained for genotype analysis. Baseline characteristics (age, gender, admission APACHE II score (KNAUS W A. et al. Crit. Care Med. (1985) 13:818-829), together with medical vs. surgical diagnosis KNAUS W A. et al. Chest (1991) 100:1619-1636) were recorded on admission to the ICU. The full cohort meeting these criteria included 1072 Caucasian subjects and 153 Asian subjects.

The Institutional Review Board at Providence Health Care and the University of British Columbia approved this study.

### Clinical Phenotype

The primary outcome variable evaluated in this study was 28-day mortality. Various organ dysfunctions were considered as secondary outcome variables. Baseline demographics recorded were age, gender, admission APACHE II score (KNAUS W A. et al. Crit. Care Med (1985) 13:818-829), and medical or surgical diagnosis on admission to the ICU (based on the APACHE III diagnostic codes) (KNAUS W A. et al. Chest (1991) 100:1619-1636) (TABLE 2B).

TABLE 2B
Baseline characteristics key.
Baseline Key

| | |
|---|---|
| AGE | Given In Years |
| GENDER | Percentage of Male Subjects |
| APACHE II | APACHE II score |
| % SURGICAL | The % of Subjects with a SURGICAL |
| | ICU admitting diagnosis |
| SS.ADMIT | Septic shock upon admission |

After meeting the inclusion criteria, data were recorded for each 24-hour period (8 am to 8 am) for 28-days after ICU admission or until hospital discharge to evaluate organ dysfunction, the intensity of SIRS (Systemic Inflammatory Response Syndrome) and sepsis. Raw clinical and laboratory variables were recorded using the worst or most abnormal variable for each 24-hour period with the exception of Glasgow Coma Score, for which the best possible score for each 24-hour period was recorded. Missing data on the date of admission was assigned a normal value and missing data after day one was substituted by carrying forward the value from the previous day. When data collection for each patient was complete, all patient identifiers were removed from all records and the patient file was assigned a unique random number linked with the blood samples. The completed raw data file was used to calculate descriptive and severity of illness scores using standard definitions as described below.

Organ dysfunction was first evaluated at baseline and then daily using the Brussels score (SIBBALD W J. and VINCENT J L. Chest (1995) 107(2):522-7) (see TABLE 2A in General Methods Section). If the Brussels score was moderate, severe, or extreme dysfunction then organ dysfunction was recorded as present on that day. To correct for deaths during the observation period, we calculated the days alive and free of organ dysfunction (RUSSELL J A. et al. Crit. Care Med (2000) 28(10):3405-11 and BERNARD G R. et al. Chest (1997) 112(1):164-72) (TABLE 2C). For example, the severity of cardiovascular dysfunction was assessed by measuring days alive and free of cardiovascular dysfunction over a 28-day observation period. Days alive and free of cardiovascular dysfunction was calculated as the number of days after inclusion that a patient was alive and free of cardiovascular dysfunction over 28-days. Thus, a lower score for days alive and free of cardiovascular dysfunction indicates more cardiovascular dysfunction. The reason that days alive and free of cardiovascular dysfunction is preferable to simply presence or absence of cardiovascular dysfunction is that severe sepsis has a high acute mortality so that early death (within 28-days) precludes calculation of the presence or absence of cardiovascular dysfunction in dead subjects. Organ dysfunction has been evaluated in this way in observational studies (Russell J A. et al. Crit. Care Med (2000) 28(10):3405-11) and in randomized controlled trials of new therapy in sepsis, acute respiratory distress syndrome (BERNARD G R. et al. N Engl J Med (1997) 336(13):912-8) and in critical care (HEBERT P C. et al. N Engl J Med (1999) 340(6):409-17).

To further evaluate cardiovascular, respiratory, and renal function we also recorded, during each 24-hour period, vasopressor support, mechanical ventilation, and renal support, respectively. Vasopressor use was defined as dopamine>5 μg/kg/min or any dose of norepinephrine, epinephrine, TNF, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis).

As a cumulative measure of the severity of SIRS, the presence of two, three or four of the SIRS criteria was scored each day over the 28-day observation period SIRS was considered present when subjects met at least two of four SIRS criteria. The SIRS criteria were 1) fever (>38° C.) or hypothermia (<36° C.), 2) tachycardia (>90 beats/min in the absence of beta-blockers, 3) tachypnea (>20 breaths/min) or need for mechanical ventilation, and 4) leukocytosis (total leukocyte count>12,000/μL or <4,000/μL).

TABLE 2C
Primary and secondary outcome variables for the ICU cohort and subsets
Survival and Days alive and free (DAF) of organ dysfunction Key

| | |
|---|---|
| SURVIVAL | 28-Day Survival |
| DA | Days alive |
| PRESS.DAF | Days alive and free of any vasopressors |
| STER.DAF | Days alive and free of steroids |
| CVS.DAF | Days alive and free of cardiovascular dysfunction |
| RESP.DAF | Days alive and free of respiratory dysfunction |
| VENT.DAF | Days alive and free of mechanical ventilators |
| COAG.DAF | Days alive and free of coagulation dysfunction |
| BVR.DAF | Days alive and free of international normalized |
| | ratio >1.5 |
| RENAL.DAF | Days alive and free of any type of renal dysfunction |
| RENSUP.DAF | Days alive and free of renal support |
| LIVER.DAF | Days alive and free of any type of hepatic dysfunction |

### Selection of SNPs for Genotyping

Publicly available genotype data was queried from the International HapMap Project (www.hapmap.org) and Seattle SNPs (http://pga.gs.washington.edu/) to select a set of tag SNPs (tSNPs) in the MAP3K14 region, each having a minor allele frequency (MAF) greater than 0.05. These tSNPs were chosen using several statistical methods, including pairwise linkage disequilibrium (LD) measures (DEVLIN B. and RISCH N. Genomics (1995) 29:311-322), haplotype (STEPHENS M. et al. Am J Hum Genet. (2001) 68:978-989; and EXCOFFIER L. and SLATKIN M. Mol. Biol. Evol. (1995) 12(5):921-927) and haplotype block (HAWLEY M E. and KIDD K K. J. Heredity. (1995) 86:409-411) patterns, as well as phylogenetic (cladistic) distance metrics (HAWLEY M E. and KIDD K K. (1995)). When these methods did not yield a parsimonious conclusion, SNPs closest in physical distance to the given gene of interest were selected. Each polymorphism was genotyped in the ICU Cohort.

### Sample Analysis

### Sample Preparation

Discarded whole blood samples, stored at 4° C., were collected from the hospital laboratory. DNA was extracted from buffy coat using the QIAamp DNA Midi kit (Qiagen, Mississauga, ON, Canada). After extraction, the DNA samples were transferred to 1.5 mL cryotubes, bar coded and cross-referenced with a unique patient number and stored at −80° C.

TABLE 2D
tSNP genotyped in ICU

| Gene | tSNPs |
|---|---|
| MAP3K14 | rs7222094 |

### Illumina Genotyping

Single nucleotide polymorphisms in MAP3K14 were genotyped using the Illumina Golden Gate™ assay from 250 ng of DNA extracted from buffy coat. A list of these SNPs can be found in TABLE 2D found in the General Methods section.

### Statistical Analysis

A description of the statistical analysis used is provided for each example in the following sections.

### EXAMPLES

### Example 1

### Risk of Death and Organ Dysfunction

### Methods

### Cohort Selection

To investigate whether genotype predicts risk of death and organ dysfunction, selected subsets of the ICU cohort were used for this study. The two study groups were: ICU Caucasians with severe sepsis upon admission (n=820) and ICU subjects of all ethnicities with severe sepsis upon admission (n=1027). Of these, 646 Caucasian and 817 all ethnicities subjects were respectively genotyped for MAP3K14 rs7222094.

### Data Analysis

All data analysis was carried out using statistical packages available in R(R Core Development Group, 2005—R Development Core Team (www.R-project.org). R: A language and environment for statistical computing. Vienna, Austria. 2005). Chi-square and Kruskal-Wallis (KW) test statistics were used in conjunction with Cox proportional hazards (CPH) regression to identify significant SNP-phenotype associations, as well as to identify baseline characteristics (age, gender, admitting APACHE II score, and medical vs. surgical admitting diagnosis) requiring post-hoc, multivariate adjustment. Genetically heterogenous populations were subsetted prior to analysis to avoid confounding from potential population stratification.

### Results

### 1.1 Mitogen-Activated Protein Kinase Kinase Kinase 14 (MAP3K14)

1.1.1 MAP3K14 rs7222094

### 1.1.1.1 Severe Sepsis—Caucasians

TABLE 3.1 gives the baseline characteristics of the 646 out of the 820 Caucasian severe sepsis subjects who were successfully genotyped at MAP3K14 rs7222094. No significant differences were detected between the two genotype groups on admission to the ICU.

TABLE 3.1
Baseline characteristics of a cohort of Caucasian Subjects with severe sepsis by genotype
of mitogen-activated protein kinase kinase kinase 14 (MAP3K14) rs7222094.

| | CC | TC | TT | Combined | Test |
|---|---|---|---|---|---|
| | (N = 180) | (N = 296) | (N = 170) | (N = 646) | Statistic |
| AGE | 48.75/59.50/72.00 | 46.00/60.00/72.00 | 45.00/56.00/69.00 | 47.00/59.00/71.00 | F = 1.44 d.f. = 2,643 P = 0.238 |
| SEX | 59% (107) | 69% (204) | 65% (110) | 65% (421) | X\hat{ }2 = 4.45 d.f. = 2 P = 0.108 |
| APACHEII | 17.00/23.50/30.25 | 18.00/23.00/29.00 | 18.00/23.00/28.00 | 18.00/23.00/29.00 | F = 0.28 d.f. = 2,643 P = 0.759 |
| SURGICAL | 31% (56) | 27% (80) | 26% (44) | 28% (180) | X\hat{ }2 = 1.38 d.f. = 2 P = 0.502 |
| SS.ADMIT | 74% (134) | 73% (216) | 75% (127) | 74% (477) | X\hat{ }2 = 0.22 d.f. = 2 P= 0.898 |

For age and APACHE II score, data is given as 25^{th} percentile/median/75^{th} percentile. For all other variables, data is given as % (N survived/N total).

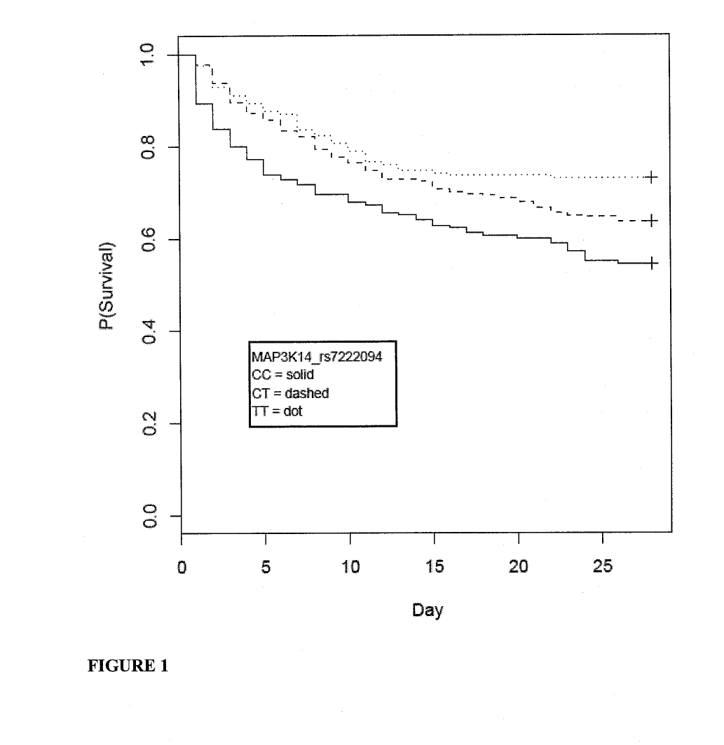

FIG. 1 and TABLE 3.2 summarize important SNP-phenotype associations. Subjects with MAP3K14 rs7222094 TT genotype showed a significantly greater survival (P=0.002), significantly more days alive (P<0.001) and days alive and free of liver failure (P<0.001), days alive and free of inotropes (P=0.002), days alive and free of pressors (P=0.01) and days alive and free of renal support (P=0.0157). Subjects who carried the MAP3K14 rs7222094 TT genotype also showed a strong trend for more days alive and free of cardiovascular dysfunction (P=0.058), days alive and free of respiratory dysfunction and of days alive and free of the use of a ventilator (P=0.093). These findings indicate that these patients who have who carry the MAP3K14 rs7222094 TT genotype have less need of inotrope and vasopressor therapy and have a lower risk of organ dysfunction (cardiovascular, coagulation, renal, hepatic and neurological).

TABLE 3.2
Days alive and free of organ dysfunction (DAF) by genotype of mitogen-activated protein kinase
kinase kinase 14 (MAP3K14) rs7222094 in a cohort of Caucasian subjects with severe sepsis.

| | CC | TC | TT | Combined | Test |
|---|---|---|---|---|---|
| | (N = 180) | (N = 296) | (N = 170) | (N = 646) | Statistic |
| SURV | 54% (98) | 64% (188) | 73% (124) | 63% (410) | X\hat{ }2 = 12.9 d.f. = 2 P = 0.002 |
| DA | 5.0/28.0/28.0 | 11.0/28.0/28.0 | 13.5/28.0/28.0 | 10.0/28.0/28.0 | F = 7.05 d.f. = 2,643 P < 0.001 |
| CVS.DAF | 0.75/6.00/14.00 | 3.00/9.00/14.00 | 3.00/8.00/14.00 | 2.00/8.00/14.00 | F = 2.85 d.f. = 2,643 P = 0.058 |
| RESP.DAF | 1.00/14.50/25.00 | 2.00/17.00/24.25 | 5.25/20.00/26.00 | 2.00/18.00/25.00 | F = 2.52 d.f. = 2,643 P = 0.081 |
| VENT.DAF | 0/12/24 | 0/13/24 | 1/19/25 | 0/15/24 | F = 2.38 d.f. = 2,643 P = 0.093 |
| COAG.DAF | 3/24/28 | 9/26/28 | 11/28/28 | 8/26/28 | F = 4.4 d.f. = 2,643 P = 0.013 |
| RENAL.DAF | 0.0/1.0/26.0 | 0.0/4.5/26.0 | 0.0/9.0/26.0 | 0.0/3.5/26.0 | F = 1.38 d.f. = 2,643 P = 0.252 |
| LIVER.DAF | 4.75/24.00/28.00 | 10.00/28.00/28.00 | 13.50/28.00/28.00 | 9.00/28.00/28.00 | F = 8.62 d.f. = 2,643 P < 0.001 |
| INR.DAF | 4.0/22.0/28.0 | 8.0/25.0/28.0 | 11.5/27.0/28.0 | 8.0/25.0/28.0 | F = 6.1 d.f. = 2,643 P = 0.002 |
| PRESS.DAF | 2/21/27 | 8/23/27 | 8/25/28 | 5/23/27 | F = 4.65 d.f. = 2,643 P = 0.01 |
| RENSUP.DAF | 4.00/22.00/28.00 | 6.00/26.00/28.00 | 9.00/28.00/28.00 | 5.75/27.00/28.00 | F = 5.01 d.f. = 2,643 P = 0.007 |

For all variables besides 28-day survival, data is given as 25^{th} percentile/median/75^{th} percentile. For 28-day survival, data is given as % (N survived/N total).

A logistic regression approach was also used to test for a statistically significant interaction between genotype and 28-day survival in Caucasians. TABLE 3.3 shows that there is a significant interaction between MAP3K14 rs7222094 genotype, and survival (P=0.000361), confirming that Caucasian subjects with the MAP3K14 rs7222094 CC genotype have decreases 28-day survival. In contrast, 28-day survival is improved in Caucasian subjects with the MAP3K14 rs7222094 TT genotype.

TABLE 3.3
A logistic regression of the CC genotype vs. CT genotype vs.
TT genotype of mitogen-activated protein kinase kinase kinase
14 (MAP3K14) rs7222094 on 28-day survival in Caucasians.

| | Estimate | Std. Error | z value | Pr(>\|z\|) |
|---|---|---|---|---|
| (Intercept) | 0.1658 | 0.1336 | 1.241 | 0.214453 |
| marker | 0.4047 | 0.1135 | 3.567 | 0.000361 |

### 1.1.1.2 Severe Sepsis—All Ethnicities

TABLE 3.4 gives the baseline characteristics of 817 of the 1027 subjects of all ethnicities with severe sepsis who were successfully genotyped at MAP3K14) rs7222094. No significant differences were detected between the genotype groups on admission to the ICU.

TABLE 3.4
Baseline characteristics of a cohort of subjects in all ethnicities with severe sepsis
by genotype of mitogen-activated protein kinase kinase kinase 14 (MAP3K14) rs7222094.

| | CC | TC | TT | Combined | Test |
|---|---|---|---|---|---|
| | (N = 281) | (N = 345) | (N = 191) | (N = 817) | Statistic |
| AGE | 48.0/61.0/73.0 | 46.0/60.0/72.0 | 45.0/57.0/69.0 | 46.0/60.0/71.5 | F = 2.11 d.f. = 2,814 P = 0.122 |
| SEX | 58% (164) | 66% (226) | 64% (122) | 63% (512) | X\hat{ }2 = 3.53 d.f. = 2 P = 0.171 |
| APACHEII | 18/24/31 | 18/23/29 | 18/23/28 | 18/23/30 | F = 1.44 d.f. = 2,814 P = 0.237 |
| SURGICAL | 29% (82) | 27% (92) | 27% (52) | 28% (226) | X\hat{ }2 = 0.51 d.f. = 2 P = 0.774 |
| SS.ADMIT | 77% (215) | 74% (254) | 76% (145) | 75% (614) | X\hat{ }2 = 0.77 d.f. = 2 P = 0.68 |

For age and APACHE II score, data is given as 25^{th} percentile/median/75^{th} percentile. For all other variables, data is given as % (N survived/N total).

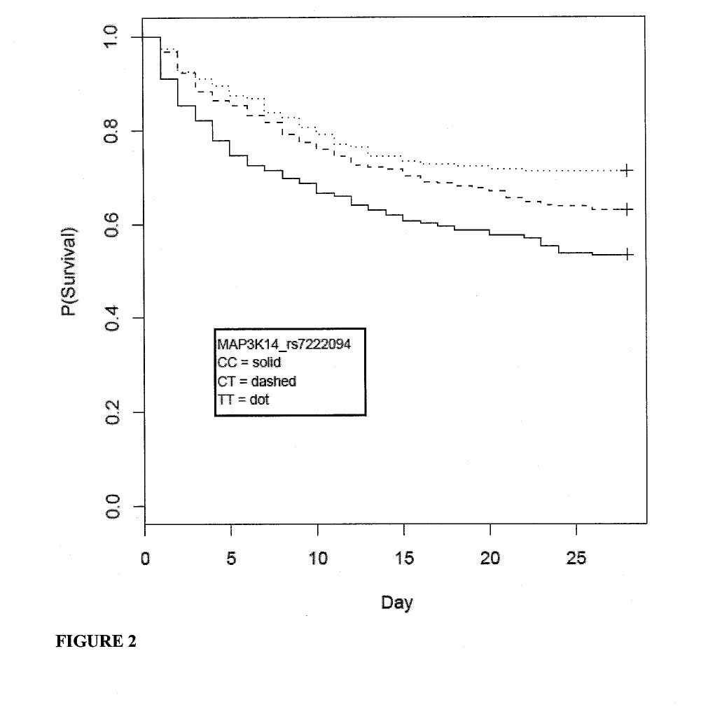

FIG. 2 and TABLE 3.5 summarize important SNP-phenotype associations. Subjects with MAP3K14 rs7222094 TT genotype showed a significantly greater survival (P<0.001), significantly more days alive (P<0.001) and days alive and free of cardiovascular dysfunction (P=0.001), days alive and free of respiratory dysfunction (P=0.037), days alive and free of the use of a ventilator (P=0.045), days alive and free of coagulation dysfunction (P=002), days alive and free of liver failure (P<0.001), days alive and free of inotropes (P<0.001), days alive and free of pressors (P=0.001) and days alive and free of renal support (P<0.001). These findings indicate that these patients who have who carry the MAP3K14 rs7222094 TT genotype have less need of inotrope and vasopressor therapy and have a lower risk of organ dysfunction (cardiovascular, coagulation, renal, hepatic and neurological).

TABLE 3.5
Days alive and free of organ dysfunction (DAF) by genotype of mitogen-activated protein kinase kinase
kinase 14 (MAP3K14) rs7222094 in a cohort of subjects in all ethnicities with severe sepsis.

| | CC | TC | TT | Combined | Test |
|---|---|---|---|---|---|
| | (N = 281) | (N = 345) | (N = 191) | (N = 817) | Statistic |
| SURV | 53% (150) | 63% (217) | 71% (136) | 62% (503) | X\hat{ }2 = 15.71 d.f. = 2 P < 0.001 |
| DA | 5/28/28 | 11/28/28 | 13/28/28 | 10/28/28 | F = 8.78 d.f. = 2,814 P < 0.001 |
| CVS.DAF | 0/5/13 | 3/8/14 | 3/8/14 | 2/7/14 | F = 6.96 d.f. = 2,814 P = 0.001 |
| RESP.DAF | 1.0/12.0/25.0 | 2.0/17.0/25.0 | 5.5/20.0/26.0 | 1.5/17.0/25.0 | F = 3.31 d.f. = 2,814 P = 0.037 |
| VENT.DAF | 0/9/24 | 0/13/24 | 1/19/25 | 0/14/24 | F = 3.1 d.f. = 2,814 P = 0.045 |
| COAG.DAF | 4/24/28 | 9/26/28 | 12/28/28 | 7/26/28 | F = 6.07 d.f. = 2,814 P = 0.002 |
| RENAL.DAF | 0/2/26 | 0/5/26 | 0/12/26 | 0/5/26 | F = 1.78 d.f. = 2,814 P = 0.169 |
| LIVER.DAF | 5/24/28 | 10/28/2028 | 13/28/28 | 8/28/28 | F = 11.77 d.f. = 2,814 P < 0.001 |
| INR.DAF | 4/21/28 | 8/25/28 | 12/27/28 | 7/25/28 | F = 7.53 d.f. = 2,814 P < 0.001 |
| PRESS.DAF | 2/19/27 | 7/23/27 | 8/25/28 | 4/23/27 | F = 6.69 d.f. = 2,814 P = 0.001 |
| RENSUP.DAF | 3/18/28 | 6/24/28 | 9/28/28 | 5/25/28 | F = 8 d.f. = 2,814 P < 0.001 |

For all variables besides 28-day survival, data is given as 25^{th} percentile/median/75^{th} percentile. For 28-day survival, data is given as % (N survived/N total).

A logistic regression approach was also used to test for a statistically significant interaction between genotype and 28-day survival. TABLE 3.6 shows that there is a significant interaction between MAP3K14 rs7222094 genotype, and survival (P=0.0000831), confirming that subjects in all ethnicities with the MAP3K14 rs7222094 CC genotype have decreases 28-day survival. In contrast, 28-day survival is improved in subjects in all ethnicities with the MAP3K14 rs7222094 TT genotype.

TABLE 3.6
A logistic regression of the CC genotype vs. CT genotype vs.
TT genotype of mitogen-activated protein kinase kinase kinase
14 (MAP3K14) rs7222094 on 28-day survival in all ethnicities.

| | Estimate | Std. Error | z value | Pr(>\|z\|) |
|---|---|---|---|---|
| (Intercept) | 0.13783 | 0.10962 | 1.257 | 0.209 |
| marker | 0.38588 | 0.09806 | 3.935 | 8.31E−05 |

### Example 2

### Response to Steroids in Severe Sepsis

### Methods

### Cohort Selection

To investigate whether genotype predicts response to steroid treatment, a subset of severe sepsis subjects treated with steroids (N=439) were compared all other subjects with severe sepsis who had not been administered steroids (intravenous hydrocortisone at about 100 mg intravenous every 8 hours) (N=588). Steroid treatment has been associated with an increase in IL-10 expression, which in turn is associated with suppression of TNF production (Denys A, Udalova I A, Smith C, Williams L M, Ciesielski C J, Campbell J, Andrews C, Kwaitkowski D, Foxwell B M. Evidence for a dual mechanism for IL-10 suppression of TNF-alpha production that does not involve inhibition of p38 mitogen-activated protein kinase or NF-kappa B in primary human macrophages J. Immunol. 2002 May 15; 168(10):4837-45. Gayo A, Mozo L, Suárez A, Tuñon A, Lahoz C, Gutiérrez C. Glucocorticoids increase IL-10 expression in multiple sclerosis patients with acute relapse. J. Neuroimmunol. 1998 May 15; 85(2):122-30).)

### Data Analysis

All data analysis was carried out using statistical packages available in R(R Core Development Group, 2005—R Development Core Team (www.R-project.org). R: A language and environment for statistical computing. Vienna, Austria. 2005). Chi-square and Kruskal-Wallis (KW) test statistics were used in conjunction with Cox proportional hazards (CPH) regression to identify significant SNP-phenotype associations, as well as to identify significantly different baseline characteristics (age, gender, admitting APACHE II score, and medical vs. surgical admitting diagnosis) requiring post-hoc, multivariate adjustment.

Using 28-day survival as the outcome variable and a chi-squared test of significance, SNP-phenotype comparisons were undertaken within and between treatment groups. We considered a by-genotype effect to be significant when two criteria were fulfilled. First, we expected an increase in 28-day survival for steroid-treated subjects compared to controls. Second, we required a p-value<0.1 for this difference in 28-day survival. When both criteria were met, we considered the allele or genotype predicting increased 28-day survival with steroid treatment to be an “improved response genotype” (IRG). Only IRG polymorphisms were evaluated for organ dysfunction results and were compared between steroid-treated subjects and controls using a Kruskal-Wallis test.

### Results

### 2.1 Mitogen-Activated Protein Kinase Kinase Kinase 14 (MAP3K14)

2.1.1 MAP3K14 rs7222094

It was unknown whether SNPs within the MAP3K14 gene and those regions immediately upstream and downstream would be associated with the response to steroids. It was found that MAP3K14 rs7222094 can be used to predict response (28-day survival) to steroid treatment in subjects with severe sepsis. Of 439 steroid-treated and 588 control subjects with severe sepsis, 355 and 462 were respectively genotyped for MAP3K14 rs7222094. Baseline characteristics for subjects with genotypes are shown in Table 4.1 and Table 4.2.

TABLE 4.1
Baseline characteristics of a group of steroid-treated severe sepsis subjects by genotype
of mitogen-activated protein kinase kinase kinase 14 (MAP3K14) rs7222094.

| | CC | TC | TT | Combined | Test |
|---|---|---|---|---|---|
| | (N = 122) | (N = 159) | (N = 74) | (N = 355) | Statistic |
| AGE | 46.25/58.00/69.00 | 46.50/62.00/72.00 | 43.00/57.00/67.75 | 46.50/60.00/70.50 | F = 1.35 d.f. = 2,352 P = 0.262 |
| SEX | 55% (67) | 62% (98) | 55% (41) | 58% (206) | X\hat{ }2 = 1.54 d.f. = 2 P = 0.462 |
| APACHEII | 21.00/26.00/33.00 | 19.00/25.00/30.00 | 19.25/24.00/30.75 | 20.00/25.00/31.00 | F = 1.61 d.f. = 2,352 P = 0.202 |
| SURGICAL | 25% (30) | 23% (36) | 20% (15) | 23% (81) | X\hat{ }2 = 0.49 d.f. = 2 P = 0.781 |
| SS.ADMIT | 84% (103) | 82% (130) | 76% (56) | 81% (289) | X\hat{ }2 = 2.35 d.f. = 2 P = 0.308 |

For age and APACHE II score, data is given as 25^{th} percentile | median | 75^{th} percentile. For all other variables, data is given as % (N/N total).

TABLE 4.2
Baseline characteristics or a steroid untreated control severe sepsis subjects by
genotype of mitogen-activated protein kinase kinase kinase 14 (MAP3K14) rs7222094.

| | CC | TC | TT | Combined | Test |
|---|---|---|---|---|---|
| | (N = 159) | (N = 186) | (N = 117) | (N = 462) | Statistic |
| AGE | 49.50/64.00/74.50 | 45.25/59.00/72.00 | 46.00/57.00/69.00 | 46.00/61.00/72.00 | F = 2.46 d.f. = 2,459 P = 0.086 |
| SEX | 61% (97) | 69% (128) | 69% (81) | 66% (306) | X\hat{ }2 = 2.97 d.f. = 2 P = 0.227 |
| APACHEII | 15/22/30 | 16/22/27 | 17/21/26 | 16/22/28 | F = 0.11 d.f. = 2,459 P = 0.892 |
| SURGICAL | 33% (52) | 30% (56) | 32% (37) | 31% (145) | X\hat{ }2 = 0.27 d.f. = 2 P = 0.873 |
| SS.ADMIT | 70% (112) | 67% (124) | 76% (89) | 70% (325) | X\hat{ }2 = 3.04 d.f. = 2 P = 0.218 |

For age and APACHE II score, data is given as 25^{th} percentile | median | 75^{th} percentile. For all other variables, data is given as % (N/N total).

TABLE 4.3 and TABLE 4.4 show 28-day survival and organ dysfunction data by MAP3K14 rs7222094 genotype for steroid-treated and control subjects respectively. TABLE 4.5 shows the differences in survival and measures of organ dysfunction between by MAP3K14 rs7222094 genotype between steroid-treated and control subjects.

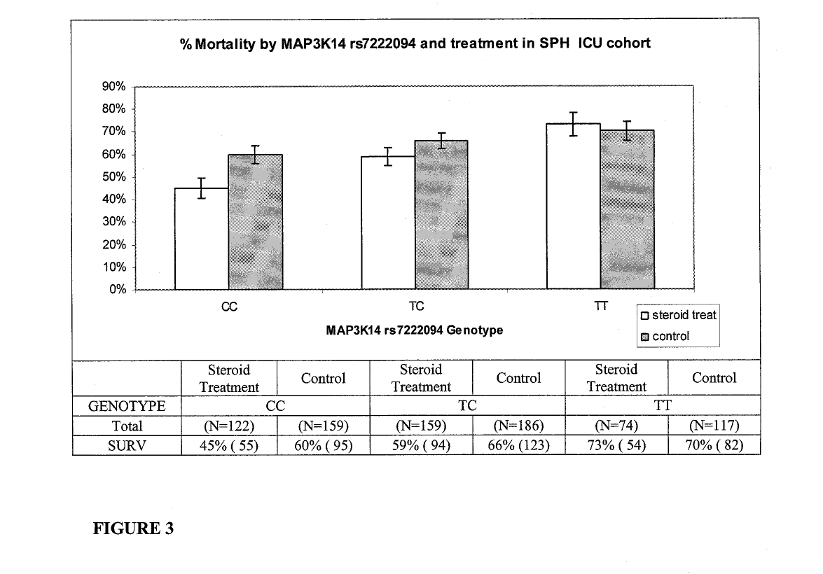

In general, FIG. 3 and TABLE 4.5 shows that steroid-treated subjects with MAP3K14 rs7222094 CC had lower 28-day survival and more organ dysfunction than controls as evidenced by negative values for the MAP3K14 rs7222094 CC subjects in the DELTA column. In contrast, steroid-treated subjects with the MAP3K14 rs7222094 TT genotype had increased survival and improved organ function (shown by greater DAF) compared to controls as demonstrated by the generally positive values in DELTA column. There was a small increase in 28-day survival of subjects with the MAP3K14 rs7222094 TT genotype in steroid-treated subjects (66%) compared to controls (59%).

TABLE 4.3
A response association of mitogen-activated protein kinase kinase kinase 14 (MAP3K14)
rs7222094 in a group of ICU severe sepsis subjects treated with steroids.

| | CC | TC | TT | Combined | Test |
|---|---|---|---|---|---|
| | (N = 122) | (N = 159) | (N = 74) | (N = 355) | Statistic |
| SURV | 45% (55) | 59% (94) | 73% (54) | 57% (203) | X\hat{ }2 =15.08 d.f. = 2 P < 0.001 |
| DA | 6.0/23.0/28.0 | 10.0/28.0/28.0 | 17.5/28.0/28.0 | 9.0/28.0/28.0 | F = 7.38 d.f. = 2,352 P < 0.001 |
| CVS.DAF | 0.00/5.00/12.75 | 1.00/8.00/13.00 | 3.25/9.00/14.00 | 1.00/7.00/14.00 | F = 3.69 d.f. = 2,352 P = 0.026 |
| RESP.DAF | 0.00/8.00/22.00 | 1.00/12.00/22.50 | 6.25/19.00/25.00 | 1.00/12.00/23.00 | F = 4.3 d.f. = 2,352 P = 0.014 |
| VENT.DAF | 0.00/5.00/20.75 | 0.00/7.00/21.00 | 1.00/18.00/24.00 | 0.00/9.00/22.00 | F = 4 d.f. = 2,352 P = 0.019 |
| COAG.DAF | 3.0/15.5/28.0 | 8.0/24.0/28.0 | 11.5/27.5/28.0 | 6.0/23.0/28.0 | F = 5.8 d.f. = 2,352 P = 0.003 |
| RENAL.DAF | 0.0/0.0/25.0 | 0.0/1.0/25.0 | 0.0/6.5/26.0 | 0.0/1.0/26.0 | F = 1.42 d.f. = 2,352 P = 0.242 |
| LIVER.DAF | 5.00/19.50/28.00 | 9.00/28.00/28.00 | 16.75/28.00/28.00 | 8.00/27.00/28.00 | F = 8.96 d.f. = 2,352 P < 0.001 |
| INR.DAF | 4.00/18.00/28.00 | 8.00/25.00/28.00 | 13.75/27.00/28.00 | 7.00/24.00/28.00 | F = 6.58 d.f. = 2,352 P = 0.002 |
| PRESS.DAF | 0.25/12.00/26.00 | 6.00/18.00/26.00 | 8.25/24.00/28.00 | 2.50/19.00/27.00 | F = 6.35 d.f. = 2,352 P = 0.002 |
| RENSUP.DAF | 3.0/14.0/28.0 | 3.0/19.0/28.0 | 7.5/28.0/28.0 | 4.0/19.0/28.0 | F = 6.29 d.f. = 2,352 P = 0.002 |

For 28-day survival, data is given as % (N survived/N total). For all variables besides 28-day survival, data is given as 25^{th} percentile/median/75^{th} percentile.

TABLE 4.4
A response association of mitogen-activated protein kinase kinase kinase 14 (MAP3K14)
rs7222094 in a control group of ICU severe sepsis subjects not treated with steroids.

| | CC | TC | TT | Combined | Test |
|---|---|---|---|---|---|
| | (N = 159) | (N = 186) | (N = 117) | (N = 462) | Statistic |
| SURV | 60% (95) | 66% (123) | 70% (82) | 65% (300) | X\hat{ }2 = 3.36 d.f. = 2 P = 0.187 |
| DA | 5/28/28 | 12/28/28 | 13/28/28 | 10/28/28 | F = 2.51 d.f. = 2,459 P = 0.082 |
| CVS.DAF | 1.0/6.0/13.0 | 4.0/8.5/15.0 | 3.0/8.0/14.0 | 2.0/7.0/15.0 | F = 3.89 d.f. = 2,459 P = 0.021 |
| RESP.DAF | 1/20/27 | 4/19/26 | 5/21/26 | 2/21/26 | F = 0.27 d.f. = 2,459 P = 0.761 |
| VENT.DAF | 0.00/16.00/26.00 | 1.25/18.00/25.00 | 1.00/20.00/26.00 | 0.00/18.50/25.00 | F = 0.29 d.f. = 2,459 P = 0.746 |
| COAG.DAF | 4.50/27.00/28.00 | 11.25/27.00/28.00 | 13.00/28.00/28.00 | 9.00/27.00/28.00 | F = 1.31 d.f. = 2,459 P = 0.271 |
| RENAL.DAF | 0.0/4.0/26.0 | 0.0/10.5/26.0 | 0.0/12.0/27.0 | 0.0/8.0/26.0 | F = 0.45 d.f. = 2,459 P = 0.641 |
| LIVER.DAF | 5.00/26.00/28.00 | 10.25/28.00/28.00 | 12.00/28.00/28.00 | 9.00/28.00/28.00 | F = 4.01 d.f. = 2,459 P = 0.019 |
| INR.DAF | 4/24/28 | 9/26/28 | 12/27/28 | 8/26/28 | F = 2.01 d.f. = 2,459 P = 0.135 |
| PRESS.DAF | 2.00/23.00/28.00 | 10.00/25.00/28.00 | 8.00/26.00/28.00 | 5.75/25.00/28.00 | F = 1.48 d.f. = 2,459 P = 0.23 |
| RENSUP.DAF | 4.00/23.00/28.00 | 7.25/28.00/28.00 | 9.00/28.00/28.00 | 6.00/28.00/28.00 | F = 2.51 d.f. = 2,459 P = 0.082 |

For 28-day survival, data is given as % (N survived/N total). For all variables besides 28-day survival, data is given as 25^{th} percentile | median | 75^{th} percentile.

TABLE 4.5
Difference in response association of mitogen-activated protein kinase kinase kinase
14 (MAP3K14) rs7222094 between cases (steroid-treated group) (Treat) and controls (steroid
untreated controls) (Cont) of ICU subjects diagnosed with severe sepsis.

| | CC | | | TC | | | TT | | |
|---|---|---|---|---|---|---|---|---|---|
| | treat | cont | | treat | cont | | treat | cont | |
| | (N = 122) | (N = 159) | Delta | (N = 159) | (N = 186) | Delta | (N = 74) | (N = 117) | Delta |
| SURV | 45% (55) | 60% (95) | −15.0 | 59% (94) | 66% (123) | −7.0 | 73% (54) | 70% (82) | 3.0 |
| DA | 23 | 28 | −5.0 | 28 | 28 | 0.0 | 28 | 28 | 0.0 |
| CVS.DAF | 5 | 6 | −1.0 | 8 | 8.5 | −0.5 | 9 | 8 | 1.0 |
| RESP.DAF | 8 | 20 | −12.0 | 12 | 19 | −7.0 | 19 | 21 | −2.0 |
| VENT.DAF | 5 | 16 | −11.0 | 7 | 18 | −11.0 | 18 | 20 | −2.0 |
| COAG.DAF | 15.5 | 27 | −11.5 | 24 | 27 | −3.0 | 27.5 | 28 | −0.5 |
| RENAL.DAF | 0 | 4 | −4.0 | 1 | 10.5 | −9.5 | 6.5 | 12 | −5.5 |
| LIVER.DAF | 19.5 | 26 | −6.5 | 28 | 28 | 0.0 | 28 | 28 | 0.0 |
| INR.DAF | 18 | 24 | −6.0 | 25 | 26 | −1.0 | 27 | 27 | 0.0 |
| PRESS.DAF | 12 | 23 | −11.0 | 18 | 25 | −7.0 | 24 | 26 | −2.0 |
| RENSUP.DAF | 14 | 23 | −9.0 | 19 | 28 | −9.0 | 28 | 28 | 0.0 |

For all variables besides 28-day survival, data is presented as medians. For 28-day survival, data is presented as %(N survived/N total).

A logistic regression approach was used to test for a statistically significant interaction between genotype and steroid use as predicted by 28-day survival TABLE 4.6 shows that there is a trend towards an interaction between MAP3K14 rs7222094 genotype, steroid treatment and survival (P=0.07452), confirming that treatment with steroids decreases 28-day survival in MAP3K14 rs7222094 CC subjects. In contrast, 28-day survival for steroid-treated subjects with the MAP3K14 rs7222094 TT genotype is improved compared with controls. In addition, steroid-treated MAP3K14 rs7222094 CC subjects had significantly worse survival that untreated MAP3K14 rs7222094 CC subjects (15%, p=0.02023; Pearson's Chi-square).

TABLE 4.6
Interaction between steroid use vs. no steroid use
(controls) and CC genotype vs. CT genotype vs. TT
genotype of mitogen-activated protein kinase kinase
kinase 14 (MAP3K14) rs7222094 on 28-day survival.

| | Estimate | Std. Error | z value | Pr(>\|z\|) |
|---|---|---|---|---|
| (Intercept) | 0.4098 | 0.1483 | 2.763 | 0.00573 |
| marker | 0.2324 | 0.1285 | 1.809 | 0.07049 |
| STER | −0.6165 | 0.2231 | −2.763 | 0.00573 |
| marker:STER | 0.358 | 0.2007 | 1.783 | 0.07452 |

1. A method for obtaining a prognosis for a subject having, or at risk of developing, an inflammatory condition, the method comprising determining a genotype of said subject which includes one or more polymorphic sites in the subject's MAP3K14 gene sequence, wherein said genotype is indicative of an ability of the subject to recover from the inflammatory condition and wherein the polymorphic site is at rs7222094 or a polymorphic site in linkage disequilibrium thereto.

2. The method of claim 1, wherein a subject having an rs7222094CC or rs7222094CT genotype or a polymorphic site in linkage disequilibrium thereto, is at risk of having a poor outcome from the inflammatory condition.

3. The method of claim 1, wherein a subject having an rs7222094TT genotype or a polymorphic site in linkage disequilibrium thereto, is at reduced risk of having a poor outcome from the inflammatory condition.

4. The method of claim 1, wherein said genotype is determined using one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

5. The method of claim 1, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

6. The method of claim 1, wherein the inflammatory condition is selected from one or more of: SIRS, sepsis, septic shock and cardiogenic shock.

7. A method for selecting a group of subjects for determining the efficacy of a candidate drug known or suspected of being useful for the treatment of an inflammatory condition, the method comprising determining a genotype at one or more polymorphic sites in the subject's MAP3K14 gene sequence for each subject, wherein said genotype is indicative of the subject's ability to recover from the inflammatory condition and sorting subjects based on their genotype, wherein the polymorphic site rs7222094 or a polymorphic site in linkage disequilibrium thereto.

8. The method of claim 7 further comprising, administering the candidate drug to the subjects or a subset of subjects and determining each subject's ability to recover from the inflammatory condition.

9. The method of claim 8, further comprising comparing subject response to the candidate drug based on genotype of the subject.

10. A method of treating an inflammatory condition in a subject in need thereof, the method comprising administering to the subject an anti-inflammatory agent or an anti-coagulant agent, wherein said subject has an improved response genotype in their MAP3K14 gene sequence, wherein the improved response genotype is selected from rs7222094TT or a polymorphic site in linkage disequilibrium thereto.

11. A method of treating an inflammatory condition in a subject in need thereof, the method comprising selectively not administering an anti-inflammatory agent or an anti-coagulant agent to the subject, wherein said subject has an adverse response genotype in their MAP3K14 gene sequence, wherein the adverse response genotype is selected from rs7222094CC or rs7222094CT or a polymorphic site in linkage disequilibrium thereto.

12. A method of selecting a subject for the treatment of an inflammatory condition with an anti-inflammatory agent or an anti-coagulant agent, comprising the step of identifying a subject having an improved response genotype in their MAP3K14 gene sequence selected from rs7222094TT or a polymorphic site in linkage disequilibrium thereto, wherein the identification of a subject with the improved response genotype is predictive of increased responsiveness to the treatment of the inflammatory condition with the anti-inflammatory agent or the anti-coagulant agent.

13. A method of selecting a subject for the treatment of an inflammatory condition, comprising the step of identifying a subject having an adverse response genotype in their MAP3K14 gene sequence, selected from rs7222094CC or rs7222094CT or a polymorphic site in linkage disequilibrium thereto, wherein the subject is selectively not treated with an anti-inflammatory agent or an anti-coagulant agent, wherein the identification of a subject with the adverse response genotype is predictive of decreased responsiveness to the treatment of the inflammatory condition with the anti-inflammatory agent or the anti-coagulant agent.

14. The method of claim 12 or 13, wherein the anti-inflammatory agent or the anti-coagulant agent is a TNF inhibitor.

15. The method of claim 14, wherein the TNF inhibitor is selected from one or more of an antagonist of TNF, an aptamer that binds to TNF, an aptamer that binds to a TNF receptor, a soluble TNF receptor, an anti-TNF antibody, and an anti-TNF antibody fragment.

16. The method of claim 15, wherein the anti-TNF antibody, and the anti-TNF antibody fragment are selected from one or more of the following: infliximab; adalumimab; ovine polyclonal anti-TNF Fab fragment; certolizumab; and afelimomab.

17. The method of claim 15, wherein the soluble TNF receptor is selected from one or more of: etanercept; lenerecept; and PEG-p55sTNF-receptor I monomer.

18. The method of claim 12 or 13, wherein the anti-inflammatory agent or the anti-coagulant agent is a steroid.

19. The method of claim 18, wherein the steroid is selected from one or more of corticosteroids, cortisone, hydrocortisone, dexamethasone, fludrocortisone, triamcinolone, prednisone, methylprednisolone and prednisolone.

20. The method claim 12 or 13, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

21. The method of claim 20, wherein the inflammatory condition is selected from one or more of: SIRS, sepsis, septic shock and cardiogenic shock.

22.-31. (canceled)
